(19) United States
(12) Patent Application Publication
(10) Pub. No.: US 2006/0147051 A1
(43) Pub. Date: Jul. 6, 2006

(54) Audio system

(76) Inventors: Brian Douglas Smith, London (GB); Martin Geoffrey Aston, York (GB)

Correspondence Address: Thomas E Sisson;Jackson Walker, 112 E Pecan St, Suite 2100, San Antonio, TX, 78205, US

(21) Appl. No.: 10/559,546

(22) PCT Filed: Jun. 2, 2004
(86) PCT No.: PCT/GB04/02361

§ 371 Date: Dec. 2, 2005

(30) Foreign Application Priority Data
Jun. 2, 2003 (GB) 0312567.1
Dec. 23, 2003 (GB) 0329808.0

(51) Int. Cl. A61F 11/06; H03B 29/00; G10K 11/16

(52) U.S. Cl. 381/71.2; 381/71.9

(57) ABSTRACT

An audio system comprises, according to one aspect of the invention, an audio frequency actuator coupled to a body so as to cause the body to radiate sound when an audio signal is supplied to the actuator by a controller, and an acoustic sensor coupled to said body or to an adjacent body so as to sense acoustic vibrations in said body, the sensor being connected to the controller, whereby the controller is arranged to change the audio signal supplied to the actuator according to the vibrations sensed by the sensor. Another aspect provides an audio system comprising an audio frequency actuator coupled to a body so as to cause the body to radiate sound when an audio signal is supplied to the actuator by a controller, and an acoustic sensor coupled to said body or to an adjacent body so as to sense acoustic vibrations in said body, the sensor being connected to the controller, whereby the controller is arranged to detect predetermined characteristics of the sensed acoustic vibrations and to output an alerting signal in response thereto.

### FIELD OF THE INVENTION

This invention relates to an audio system. In one aspect, the invention relates to the conversion of an otherwise non-responsive body, for example a rigid or flexible panel, by the addition of high-force, wide-band acoustic actuators to create a smart surface that can emit and/or receive acoustic signals for the purpose of providing discreet audio or noise masking, or for sensing attack for security purposes. The audio system of this invention can be described as providing a single smart surface with Its associated acoustic locus properties or an enclosed volume when a multiplicity of such panels is activated.

The volume to be influenced can be either a singular or multiple arrangements of smart surfaces, fixed or moving.

An additional aspect of this invention also relates to the control of noise levels emitted from these smart surfaces or structural elements to avoid environmental noise pollution and create areas (or volumes) of sound masking or higher levels of noise control from the converted surfaces or structural elements.

### BACKGROUND OF THE INVENTION

Flat panel loudspeakers are well-known, and typically consist of a rigid panel of laminated plastics, card or wood with an acoustic transducer attached to one face of the panel. Typical transducers are moving coil electromagnetic devices or piezoelectric devices.

Similarly traditional cone based directional speakers are well known for reproducing audio signals or masking signals to apparently reduce the effect of ambient noise.

Magnetostrictive devices are also known, for example of the type described and claimed in our co-pending International Patent Application PCT/GB02/01111. These devices can be used particularly when larger panels require to be activated to sound. The effect of sounding a large panel by means of a magnetostrictive actuator is to distribute the sound waves through the panel, as has been described in our co-pending application, as a distributed planar signal. The effect of sounding a large panel by means of a magnetostrictive actuator is to distribute the sound waves through the panel, as has been described in our co-pending application, as a distributed planar signal. This type of signal differs from directional sound in as much as the distribution of the vibrations distribute themselves across the entire extent of the panel or structure reducing the throw but creating a flatter more consistent audio output across the entire extent of the driven item.

In a permanent or temporary building, it is known that there are multiple audio and security systems installed for many different purposes; these include audio reproduction, public announcement, supplementary emergency announcement, multi-media, and presentation amplification. Some installations also include active sound masking systems or passive sound insulation and privacy screening panels or active audio systems. Additionally, anti-intruder, counter-espionage or vandalism-detection devices are frequently fitted particularly at low or easily accessible windows.

Audio reproduction is traditionally achieved using multiple cone speakers or flat panels.

Multi media is normally achieved by installing a dedicated cone speaker system.

Public announcement is traditionally achieved using multiple cone speakers or flat panels sometimes using the audio reproduction system.

Emergency announcement is traditionally achieved using multiple cone speakers or flat panels sometimes using the audio reproduction system.

Active sound masking is frequently achieved by accurately positioning a cone speaker above a personal area, or in the top corners of a room focussing down into the centre of the room where people normally congregate for example around a table. Flat speakers can be used for this purpose also. In most cases the installation is dedicated to this purpose.

Active privacy screening is normally achieved using multiple cone speakers or flat panels often dedicated to this purpose.

Passive sound insulation is commonly used in buildings to reduce the transmission of sound from one room or area to another.

Perimeters anti intruder devices are usually positioned on windows and doors and detect movement or vibration if disturbed.

Anti vandalism systems normally include video cameras, infrared lighting and intruder devices designed to detect movement.

Counter espionage issues are significant where relevant and would be the subject of additional passive security systems, i.e. window shutters or screens.

In some countries, Sweden for example, CCTV monitoring of individuals is not permitted unless it is known that the individuals are committing an offence or breaking the law.

As can be seen in many buildings the fragmentation of the systems design often results in a multiplicity of audio and screening systems, cameras, and detection devices often resulting in excessive costs and wasted resources.

New legislation relating to noise and its control in building standards in the US (HIPPA privacy standard) and in Europe introduce demands that combine some or all of the various functionality described above into one installation classification, offering an opportunity to create a new approach to satisfy the varying needs of architects and specifiers and construction contracting companies.

This is also relevant to the control of noise in highly populated noisy environments such as call centres or night clubs where being able to make phone calls requires privacy and privacy, as well as masking to ensure that the recipient on the other end of the line can hear what you are saying.

Additionally, new European disability legislation relating to information communication, particularly to people with impaired visual sense, imposes new standards on those providing information and direction in public places. It is understood that this new legislation will challenge local authorities, exhibition organisers and public space organisers to create a new approach to provide an audio output to those provided for in the legislation.

It can be seen that the approach of converting surfaces and structures that were not originally designed to transmit audio signals into audio emitting surfaces or structures emitting positive audio (voice music etc) or masking or screening signals can have many advantages from a commercial, legislative or installation point of view, as well as from the end user's or customer's point of view.

In the use of loudspeakers or panel acoustic emitters to provide information in public spaces it is usual that the sound emitted from the panel or loudspeaker is at a constant volume, or may be turned up and down manually in response to complaints or requests. This is inconvenient and results usually from variations in the ambient noise level so that the emitted sound will at different times appear either too loud or not loud enough. If a loudspeaker is used as the emitter the point source nature of this emitter means that the sound is often projected beyond the area in which it is required and creates a noise nuisance.

It is well known that automatic gain control (AGC) circuits have been used in cars to increase the volume from the radio as the car goes faster and the ambient noise level increases. It is also known that signal compression has been used to reduce the dynamic range of radio output in car radios so that the quiet pieces of music can still be heard above high ambient noise levels, without making the louder pieces In the music too loud. Both of these mechanisms are designed to avoid the driver having to constantly adjust the volume controls. It has been found that point source emitters, loudspeakers, cannot usefully be controlled in this way unless used in a tightly defined volume of space (for example a set of headphones). It is well known that that sound needs to only be a small number of decibels higher than the ambient noise level to be noticed (at the lower end of the range) and to be heard and understood (at the upper end of the range).

Our PCT application PCT/GB02/01 111 describes an application for the use of actuators, and more particularly magnetostrictive actuators, to drive panels to be used as advertising or information media, for example kiosks in shops, the shop window itself, or train and bus panels and windows.

### SUMMARY OF THE INVENTION

This invention relates to a system solution in which the surfaces, panels and structural elements that make up buildings and vehicles, and a multiplicity of products used in day to day life such as signs, tables, bars, walls, dividers, screens desks, cupboards and cabinets, can be acoustically activated by either single or multiple actuators attached to the panels themselves or the structure that supports the panels, either by direct stimulation using an audio signal, or by creating an audio signal in response to a sensing system, thereby creating a smart surface to achieve any number of levels of functionality, but not necessarily be limited to all, as described herein.

According to the Invention, there is provided an audio system, for example for use in enclosed spaces, comprising an audio frequency actuator coupled to a panel, for example within or defining said enclosed space, so as to cause the panel to radiate sound when an audio signal is supplied to the actuator by a controller, and an acoustic sensor coupled to said panel or to an adjacent panel so as to sense acoustic vibrations in said panel, the sensor being connected to the controller, whereby the controller is arranged to change the audio signal supplied to the actuator according to the vibrations sensed by the sensor.

Another aspect of the invention provides an audio system, comprising an audio frequency actuator coupled to a panel so as to cause the panel to radiate sound when an audio signal is supplied to the actuator by a controller, and an acoustic sensor coupled to said panel or to an adjacent panel so as to sense acoustic vibrations in said panel, the sensor being connected to the controller, whereby the controller is arranged to detect predetermined characteristics of the sensed acoustic vibrations and to output an alerting signal in response thereto. This output may be acoustic, or may trigger a remote device, for example a CCTV camera or a remote alarm.

Yet another aspect of the invention relates to apparatus which provides an audio signal that has an a perceived reducing effect on the ambient noise in the vicinity, whereby it is perceived that the ambient noise is lower or less persuasive due to the proximity of the mask source to the user. This effect may be a psycho-acoustic effect due to the way that the brain perceives the mask as the dominant signal, so reducing the apparent ambient noise.

A further aspect of the invention provides a method of and apparatus for affecting noise levels in a controlled manner to enable the user to perform a task in an improved manner despite high levels of ambient noise. In one embodiment this might be in a club or bar or restaurant where a large surface emitter is used to create an audio signal that screens the ambient noise allowing conversation, phone calls to be made or simply a lower level of ambient noise to be experienced away form the general noise.

The audio frequency actuator can be integrated into the panel's construction, either internally or externally, or it could be retrospectively fitted to an existing panel.

Preferably this will include audio reproduction, public announcement, supplementary emergency announcement, multi-media, and presentation amplification. The invention may also address internal noise management, personal privacy screens, room privacy screens, external noise control, as well as possibly external counter espionage security and intruder and vandalism detection sensing.

The audio output that is seen when an actuator is coupled to a rigid panel transmits audio to both sides of the panel. The effect is approximately equal on both sides. This can be adjusted by the use of acoustic insulation whereby one side of the panel is acoustically insulated from the other by means of a soft infill material such as foam, rubber silicone or similar or a baffle or reflective materials to ensure the signal is predominantly in one direction. In some instances however the use of a two way communication is of value, for example on train windows where the audio within the carriage and on the platform would be a benefit. Similarly when a sensing system is incorporated this may be sensing on one side and acting on the other side of a panel (for example in a room in a hotel to reduce the effect of noise between rooms. Similarly in a double glazed unit the actuator may be on the inner of the front pane and sensing movement or pressure on the outer pane of glass.

Another application of this technology could be in the conversion of a structure to enable it to be able to communicate an audio signal as well as a visual image, for example a map or timetable. Increasing demand on the part of the regulators to provide for visually impaired people has increased the requirement to provide sound from a growing number of information sources normally not associated with sound generation. Traditional methods of audio reproduction will create noise pollution due to their directional characteristics and will be limited by the noise pollution that they create. The integration of an acoustically active surface or structure would allow the manufacturer of the smart surface or structure to comply with these new regulations without increasing noise pollution. In this invention the panel is driven as a consequence of the relationship to the driven frame, enabling the visual imagery to be changed at will with no need for any modification to the audio generating arrangement. The acoustic dynamics of the driven planar surface tends to show a multiplicity of small point sources that behave as one flat panel of source sound creating a lower level of sound over a wider area of audio throw.

Where others have claimed the accuracy of positioning of the actuator is critical to the performance of the panel, the massive power output of the magnetostrictive enables the actuator to be discreetly positioned within the structural element or any other suitable place that does not affect the users view (if disposable, or transparent), the visual design and character or other functionality that the sign or structure might have.

The system that provides the functionality of audio signage or audio structures in its simplest form is a signal generator that provides an audio signal to the visual panel or structure.

It can be appreciated that more complex systems, including sensing capability for noise management, and other levels of functionality can be built into this system which when combined with a digital signal processing system will allow any specific signage or signage structure to be both monitored and addressed externally or locally if the user chose to interrogate using an integrated touch panel or sensing system. In this way an attack, an enquiry (contact) or a specific noise reducing signal can be sent to the identified sign or sign structure by the processor.

If this approach was taken within say a national transport system signage structure any sign within the system fitted with a two way sensing system could be used as a communicator to any other sign in the system similarly fitted as a form of intercom or communication system. National updates could also be provided across this medium.

As each device may be fitted with its own signal amplifier, or provided for from the centralised processor, an ability to interrogate at any point in the system could be limited to audio reproduction within the immediate locus of the sign or structure, or transmitted throughout the full network.

This would have the advantage of allowing centralised digital audio to be stored for distribution throughout the whole network or on a local base only.

The audio performance of this smart surface does not rely on modifying or adding devices to the graphic material, which shall be used to convey the visual imagery only. This visual imagery may be replaceable or disposable for convenience of repeated use. According to the invention, there is provided a structure of rigid members that features an audio system that converts the otherwise non-audio designed product or structure (for example an information sign or a bus shelter or an element thereof) for use in public and commercial spaces, comprising an audio signal generator or radio link to a similar device and either a single or a multiplicity of audio frequency actuators coupled a part of the structure so as to cause the structure and the panel to radiate sound when an audio signal is supplied to the actuator by a controller. In some manifestations of the device an audio sensor is coupled to the sign or structure so as to sense audio frequency vibrations in the immediate vicinity, the sensor being connected to the controller, whereby the controller is arranged to change the audio signal supplied to the actuator according to the vibrations sensed by the sensor.

According to the invention, there is provided an acoustic device possibly but not limited to a magnetostrictive actuator acoustically coupled to a structural member or members providing multiple functionality including some but not necessarily all and not limited to, the provision of local public announcements, external public announcements, public timetable information, promotional messages, reproduction of music for entertainment purposes, and emergency announcements, as well as active sound masking of extraneous noise from the other side of the structure within the locus of the structure. Additionally anti attack, or vandalism detection may be included as a function of the actuator as it can also be tuned to intelligently sense activity on its outer face, either by reacting to sound pressure, or physical pressure (physical vandalism, or attack) or by recognising the effect of a tool or pen if scratched or written on.

The construction of the structure and the surfaces therein can vary, the common features being at least one large panel of rigid (at least in one axis) glass, plastic, wood or other sheet material of transparent, translucent or opaque finish, and a support structure of aluminium, stainless steel, painted steel, MDF glass, plastic, sheet GRC, GRP, wood or wood panelling or any other material used in the traditional signage and advertising or bus shelter market.

The smart surface or structure can be used in public, public access or commercial places where the addition of an audio signal can be advantageous for the purposes of information, advertisement or promotion by the use of high-force, wide-band acoustic actuators to create an audio signal using any number of elements of the structure of the sign for the purposes of generating a local audio signal. The input to the actuator can be any means of audio signal generator, CD, Mini disc, PC, media server or MP3, or a radio linked device that converts the signal from any type of remote signal generator. This invention can be described as a single driven element of structure with it's associated acoustic locus properties or a driven assembly of elements thereof when a multiplicity of elements are activated. The driven structure has been designed to accept the removable visual panel ensuring that the output force of the magnetostrictive drive will be sufficient to communicate a vibration through the often larger expanse of the visual panel creating a fuller audio locus around the entire structure.

The smart surface could contain printed information that shows a floor plan of an area as well as printed information intended to allow the user to locate a specific zone within the area. An overlay touch panel using some form of inductive, capacitive or other sensing device could be used to allow the use to touch a specific area of the sign in order to prompt an audible sound-bite of information. If used in conjunction with a local authority information service or timetable, instructions and information can be communicated aurally to the user.

As a system solution it is anticipated that smart surfaces and structures will have advantages In many areas of public and commercial spaces where changeable public information or aural messages for the visually disabled are required by the impending legislation, or where a deeper level of information is required to enforce or support the signage information. Where an intermittent reactive audio output is required, for example as someone approaches the sign or structure, a simple infrared or PIR detector can be integrated into the assembly that recognises the persons approach and triggers the audio message.

Where an external update of information is required to be transmitted audibly to someone in the vicinity of the smart surface or structure a combination of transmission and detection can be used to ensure that the audio signal is not transmitting to an empty space and the system can be forced to only communicate when required by someone being in the immediate detectable vicinity.

Pressure from advertisers on media companies also indicates a desire to expand the two-dimensional medium of printed advertising to encompass audible messages and information, and the massive expansion of the use of digital signage if not controlled can create a cacophony of noise in the environment that is not desirable or acceptable.

Another application for smart surfaces would be to control noise in public places such as bars and restaurants where the high level of background noise from neighbouring conversations to background music to the noise associated with food and drink serving, can make it difficult to hold a private conversation, or indeed any conversation at all for those with less acute hearing. Similarly it can be seen that the present invention provides a system for local generation and control of noise by using the magnetostrictive actuators attached to drive table tops, bars, large panels of flat rigid material mounted under or into the ceiling or floor, wall panels or other selected areas or structures that are not normally associated with audio output to create an improved noise control system. This system may be used in for example a club or bar where noise control is required to permit phone calls to be made even though the caller is in close proximity to the noise source. In this scenario a panel of material could be used to transmit a masking signal that would oppose the ambient noise. In the vicinity of the planar masking signal it would be easier to converse due to the psycho-acoustic effect of the more constant white or pink masking signal, and also it would be possible to make a phone call without the phones microphone being affected by the masking signal. In this instance the generated signal would be designed not to conflict with the sensitive microphone of the phone by using appropriate frequency signals.

Similarly in a call centre where there are a high density of operators who work in close proximity to each other (normally a six foot pitch) there is a need to mask some of the cacophony of noise generated by the operators talking at the same time. By using the magnetostrictive actuators to drive the desk or the walls (which by definition are near the caller) psycho-acoustically the operator will hear more of the consistent sound of the mask and less of the background noise, and if the frequencies are controlled to not interfere with the telephone microphone the recipient will also benefit from the reduced level of background noise perceived due to the addition of the mask.

As a system solution it is anticipated that this invention will have advantages in many areas of both domestic and commercial buildings, transportation of many types, and temporary and emergency structures including more cost effective sound installations, discreet and easier to design into schemes with additional opportunities, be more cost efficient, more reliable, addressable and future proofed to cater for new emerging higher demand building, accommodation, information, privacy standards. The invention is equally applicable to the conversion of structures such as tables, signs, bars and any other assemblies of elements of sufficient rigidity and Integrity to be converted to an audio output by the addition of a magnetostrictive actuator as described below.

According to another aspect of the invention, there is provided an acoustic device possibly but not limited to a magnetostrictive actuator acoustically coupled to a panel providing multiple functionality including some but not necessarily all and not limited to, the reproduction of music for entertainment purposes external public announcements, advertising purposes, local public announcements, external supplementary emergency announcements multi-media reproduction and presentation amplification active sound masking of extraneous noise from the other side of the panel privacy screening and room privacy within the locus of the panel or structure or localised privacy screening panels within. Additionally anti intruder, counter espionage or vandalism detection devices may be included as a function of the system's ability to monitor the condition of the panel.

The actuator also may be able to intelligently sense activity on its outer face, either by reacting to sound pressure or physical pressure (wind noise, physical vandalism, or attack) or by recognising the effect of a tool or pen if scratched or written on.

A counter espionage aspect of the system is described and claimed in our co-pending International Patent Application PCT/GB02/01111.

Detection of external pressure, sound pressure or physical pressure will preferably be via a sensor integrated into the actuator or via a remote sensor, which may be attached to the body. This sensor can either be integrated into the magnetostrictive actuator as a secondary function of the Terfenol-D material (or other GMM) or a piezoelectric device in the line of excitation, above or below the active core. It may measure actual force or it may measure the reaction force. The function of this sensor is either to sense that actual output signal or to sense in order to instruct the active device.

In a particular type of graffiti a hardened tool or similar is used to damage the surface of the glass by scratching. This has a severe visual effect on the glass and is commonly referred to as Dutch Graffiti. Another form of graffiti is with marker pens and aerosol spray paint. In each case a different acoustic signal will be detectable in the glass or surface being sensed by an integrated or separately mounted sensor. In the case of the hardened tool, a distinct signature in the region of 250 Hz to 12 KHz can be recognised and a signal sent to the controller.

In the case of anti graffiti sensing and external ambient noise sensing, this can be determined in a number of ways. Either the encoder interface box will be set up to look for one or more of the types of signals It expects (either by determining a preset sequence or selection of frequencies or another means), for example marker pen or hardened tool, or it could alternatively be set to look for the trend associated with an arising external noise such as airplane or traffic noise. It can be understood that any number of presets or learning algorithms could be used to seek and mask or screen or issue an antiphase signal to compensate or reduce the effect of the sensed noise. This could include noise from outside say a building site, noise from an adjoining room or space, noise from a specific signal such as a tool or an attack or vandalism as described or simply to create a local ambient noise screen that will mask conversation or discussion in one part of a room.

It will be understood that, while the surface or panel to be sounded might conveniently have a generally rectangular shape, for example when used as a window, the invention is not limited to the use of smart surfaces having any particular shape or purpose.

The smart surface could be a wall, or a door, or a ceiling or a floor, a vertical panel a horizontal panel, a table, an element of a larger structure, or any member that exhibits sufficiently rigid characteristics that will support audio generation using magnetostrictive actuators. A number of smart surfaces could be attached together to create a room or a volume of space general bounded by sides (for example a bus shelter). The room could be a single module as in a temporary building or self contained cabin, or it could be part of a multiplicity of rooms as in a building. The rooms could be zones within larger rooms and may not be fully contained by walls. The rooms may be static and anchored to the ground, or mobile and part of a transport system. The rooms may be subdivided with different functionality provided by alternate smart surfaces that use the same invention to provide for the acoustic environment as described.

The construction of the smart surface can vary, the common features being rigidity (at least in one axis) and size, including, glass, plastic, metal, MDF, sheet GRC, GRP, plasterboard, dry-wall, wood panelling or any other material used in the traditional building and transport industry.

The smart surface can be of composite construction using different types of material with cores and additional functionality skins, for example insulation board for temporary building construction, featuring structural Integrity outer skins, solar reflecting sheets, foam cores structural honeycomb cores, aerospace aluminium honeycomb cores corrugated cores and extruded polycarbonate or similar structural sheets, and fire insulation membranes as integral parts of its makeup. The mass of the panel can be low (small window panels) to massive 400+Kg plate glass or single skin ceiling sheets suspended or adhered to a suitable support structure.

The actuator is preferably a giant magnetostrictive material (GMM) actuator, for example of the type described and claimed in our co-pending International Patent Application PCT/GB02/01111 or a scaled up version of the same. Preferably, the actuator is bonded discreetly within the structure, or into or onto the panel or retrospectively fitted, for example by adhesive or by physical retention system of screws and mounting brackets.

The audio signal generated by any aspect of the above invention can be either a positive audio signal or an apparent negative audio signal that counters the effect of high degrees of ambient sound for a number of different purposes as explained.

Another application for the acoustic device of the invention is in the masking or perceived reduction of ambient noise. By combining with the device a sensor integrated into it or remotely mounted a distance from it or an integrated or remote microphone or the type that focuses some distance from its physical location and noise reduction or screening controller which generates an anti-phase masking or screening signal such as a white or pink noise signal corresponding to the noise received by the microphone or sensor, the device can be employed to reduce perceived noise in a room, noise coming into a room, or just affect noise in a part of a room. Similarly this will be applicable to structures that are being converted to audio generators out-with the room definition. The noise reduction may be configured to have a broad-spectrum effect or to reduce the amplitude of selected frequency bands. By configuring the layout for example, it can be arranged to activate locally when required, either triggered by ambient noise detection, or by being switched on. In this way, for example, a temporary quiet zone could be provided in an open-plan office or the like, without the need for providing relatively high-mass walls around the zone, but just by activating the ceiling, floor or adjoining wall panels. Similarly, providing patient privacy, for example in a hospital, could be achieved by activating the smart surface over or under a bed when occupied. This would also have the advantage of providing quieter conditions for a patient without the need for a separate room.

Where an intermittent reactive smart surface is required, for example window or double glazing unit that detects and reduces for example aircraft noise, or building site noise transmission into a building, it will be preferable to integrate the sensor into the actuator and mount the actuator on to the outer glass panel to facilitate more accurate sensing.

Increasing demand on the part of building regulators has also increased the requirement to provide sound insulation both from out-with and within temporary buildings. The current construction methods used are limited in the amount of noise that they can insulate against resulting in these types of buildings not being suitable for human habitation unless more material (and weight and cost) is added. The integration of an active smart surface sound screen to the external panel walls of these buildings would allow the manufacturer to comply with these new regulations without increasing the material content and weight significantly.

Active smart surfaces could be used in trains where external track noise or tunnel noise needs to be reduced or masked.

Where others have claimed the accuracy of positioning of the actuator is critical to the performance of the panel, the massive power output of the magnetostrictive enables the actuator to be discreetly positioned in the corner of the smart surface or any other suitable place that does not affect the users view (if glass) or other functionality that the smart surface might have.

The system that provides the functionality of smart surfaces in its simplest form is a signal generator that provides an audio signal to the device within or on the panel. This will reside within the encoder interface box.

It can be appreciated that more complex systems, including sensing capability functionality can be built into this system which when combined with a digital signal processing system will allow any specific smart surface to be both monitored and addressed. In this way an alarm or a specific noise reducing signal can be sent to the identified smart surface address by the processor. This could include activating external devices such as CCTV cameras or security.

It will be necessary to have a number of inputs and outputs on the encoder interface box. These are shown schematically hereinafter in FIG. 5.

It is intended that the encoder Interface box is completely universal and able to accept Inputs from a wide range of devices including analogue and digital audio, microphone and sensors.

Equally it is intended that this device can be used to monitor and affect the output on a multiplicity of devices including as described but not limited to magnetostrictive devices.

If this approach was taken within, say, an office building any window fitted with a two way sensing system could be used as a communicator to any other window similarly fitted as a form of intercom or communication system.

As each device may be fitted with its own signal amplifier, or provided for from the centralised processor, an ability to input at any point in the system could be limited to audio reproduction either within the bounded volume or a single panel locus, or transmitted throughout the full installation.

This would have the advantage of allowing centralised digital audio to be stored for distribution throughout the smart surfaces in the building or to locally addressed surfaces only.

According to another aspect of the invention, there is provided a noise control system comprising a microphone associated with a rigid surface, control means connected to the microphone to detect ambient sound and arranged to generate a control signal in anti-phase to the detected sound, and a transducer supplied with the control signal, the transducer being attached to the surface so as to cause the surface to radiate sound waves which reduce the amplitude of the ambient sound In a region adjacent to the microphone.

Preferably, the transducer comprises a giant magnetostrictive material (GMM) element whose change in strain under the influence of an audio frequency magnetic field induces sound waves into the surface.

The actuator may, for example, be an actuator of the type disclosed and claimed in our co-pending International Application WO 01/72084, although other forms of actuator will also be usable.

It has been found that by making the surface, for example a table or bar-top, radiate the sound waves, the local sound energy at the microphone is too low to cause audio feedback in the system, but the overall energy radiated is sufficient to achieve a significant noise-reduction effect. It will be appreciated that, since the anti-phase sound or masking signal radiated is based on the sound received by the microphone, the noise reduction effect is greatest near to the microphone and diminishes as one moves away from the microphone. For best effect, a plurality of microphones and associated actuators may be required.

The control means is preferably arranged to filter the sound received by the microphone, for example to filter out from the sound reduction process a voice local to the microphone, thereby permitting normal conversation to be carried on in the region of the microphone while reducing background noise.

The transducer is suitably attached to the underside of a table-top, but where this is not practical it may be attached to the upper surface or onto a rigid part of the structure. The microphone could be mounted above the surface, or below the surface with a hole being provided through the surface to permit the noise signal to be received.

This invention also provides means for varying the emitted sound level so that it is a constant level above ambient and for shaping the emitted sound envelope so that it stays within the area in which it is needed and avoids unnecessary noise pollution. It can also be used to create an envelope or curtain of white or pink noise or music signal so that private conversations are masked and cannot be overheard or zones where external noise is damped to create ‘quiet’ areas or areas where there is a perception of quiet.

### BRIEF DESCRIPTION OF THE DRAWINGS

In the drawings, which illustrate exemplary embodiments of the invention:

FIG. 1 is a diagrammatic sectional view of a combined acoustic actuator and sensor;

FIG. 2 is a similar view of an alternative configuration of actuator and sensor;

FIG. 3 is a diagrammatic sectional view through a double glazed window panel having installed therein a combined actuator and sensor according to yet another embodiment;

FIG. 4 is a similar view to that of FIG. 3, showing the actuator and sensor device installed in a wall cavity;

FIG. 5 is a block circuit diagram of a controller In accordance with the invention;

FIG. 6 is a view of a table having the noise control system installed thereon;

FIG. 7 is a sectional view through the transducer of the system In FIG. 1;

FIG. 8 is a front elevation of a sign display adapted for audio output;

FIG. 9 is a top plan view of the sign of FIG. 8; and

FIGS. 10 and 11 illustrate the actuator and contoured mounting bracket used in the audio sign shown in FIGS. 8 and 9.

### DETAILED DESCRIPTION OF THE ILLUSTRATED EMBODIMENTS

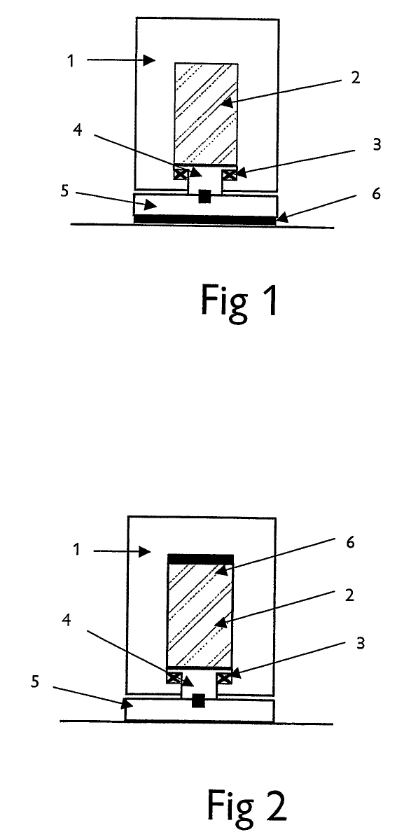

Referring first to FIGS. 1 and 2, the actuator is essentially as described in our co-pending International Patent Application PCT/GB02/01111, having a housing 1 which serves also as a reaction mass and which contains a core 2 consisting of an element of a giant magnetostrictive material (“GMM”) surrounded by an electromagnetic coil and located between permanent magnets. The element is pre-stressed by springs 3 and coupled to a pusher 4 which Is in turn connected to a foot 5, which in use is pressed against a surface into which the acoustic wave is to be transmitted. A piezoelectric sensing element 6 is incorporated into the actuator to sense acoustic signals in the surface to which the actuator is attached. In the example shown in FIG. 1, the sensing element 6 is provided on the external surface of the foot 5, while in the example shown in FIG. 2, the sensing element is located between the core 2 and the internal surface of the housing 1.

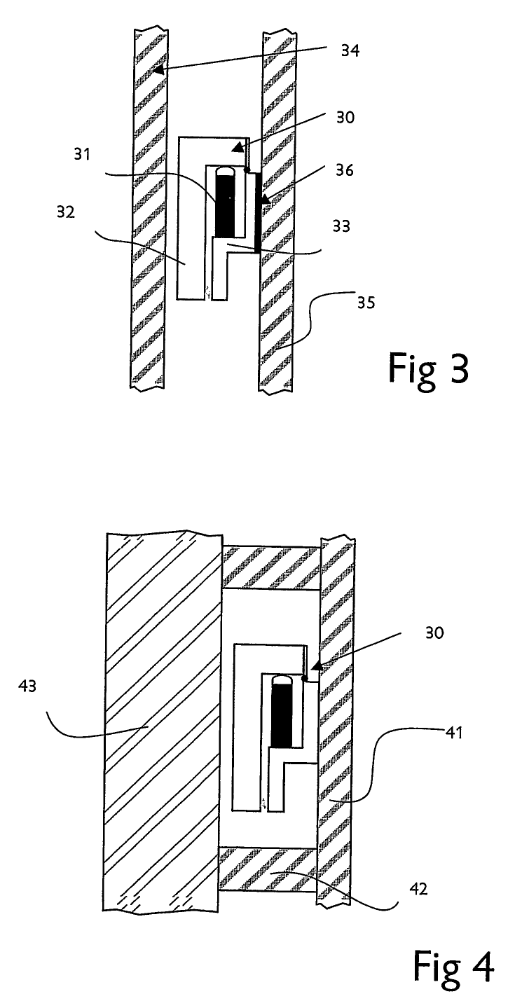

FIGS. 3 and 4 illustrate a different type of combined acoustic actuator and sensing element, in different locations in use. The actuator/sensor device 30 is of the type described in our application No PCT/GB2003/005616 and has the core 31 extending between the main body 32 of the actuator and a foot 33 pivotally mounted thereto with a spring connection holding the components 32 and 33 together and providing a pre-stress to the GMM element within the core 31. In FIG. 3, the actuator 30 is mounted between the spaced glass panes 34 and 35 of a sealed double-glazed window unit so as to act on one of the panes. The piezoelectric sensing element 36 is mounted between the foot 33 and the pane 35. In FIG. 4, the actuator 30 Is mounted on the inner face of a plasterboard wall or ceiling member 41 which can be conventionally mounted on battens 42 attached to a solid wall structure 43. Alternatively, the wall may be a studwork structure or any other building structure involving plasterboard or other boarding. The actuator 30 in FIG. 4 may be the same as that in FIG. 3, or the piezoelectric sensing element may be incorporated into the actuator, for example between the core 31 and the main body 32.

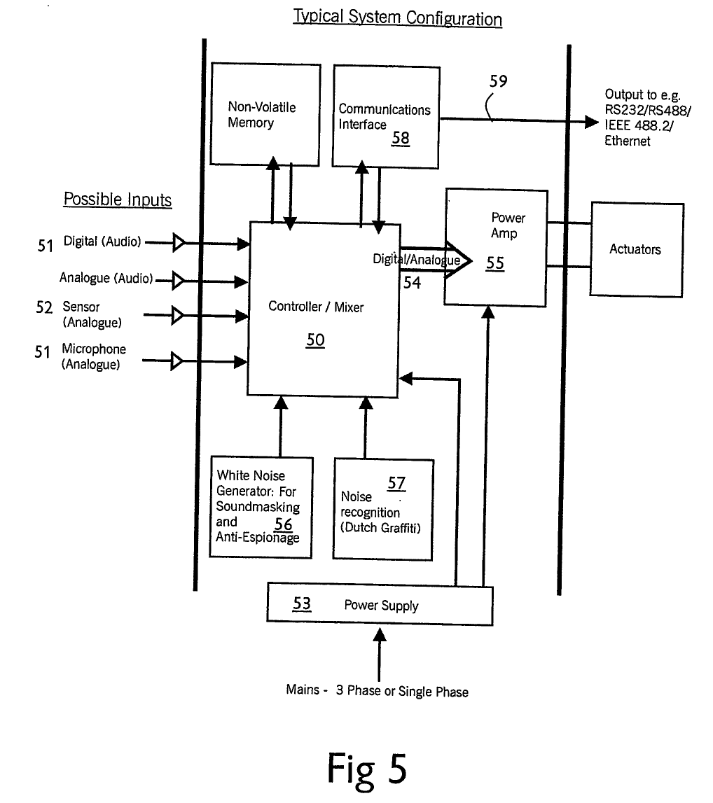

FIG. 5 illustrates a typical configuration of controller for the system of the invention. The controller includes a central processor 50 provided with audio inputs 51 for music, announcements or communications, for example, together with sensor inputs 52 connected to the sensing elements in the actuators coupled to the controller, for example those illustrated in FIGS. 1 to 4. A mains power supply 53 is included. Battery backup may also be provided. An audio output 54 is provided to a power amplifier 55. This may in turn be connected to a number of actuators in the building or vehicle, for example, or a separate feed and amplifier may be provided to individual actuators or to separate groups of actuators. A white noise generator 56 is connected to the processor to provide a sound-masking output to the actuators or to selected actuators in response to detected noise or to prevent espionage by monitoring vibrations in windows or other panels, for example. A noise recognition module 57 compares acoustic signals detected by the sensors with a library of acoustic signatures to recognise a potentially damaging attack on a panel, for example by cutting or scratching, and outputs an alarm signal in response to detection of such a signal. The alarm signal can give rise to audio output to the panel concerned and/or to adjacent panels, and a message can also be transmitted to a remote monitoring location via a communications interface 58 and communications link 59 (e.g. RS232/RS488/IEEE 488.2/Ethernet).

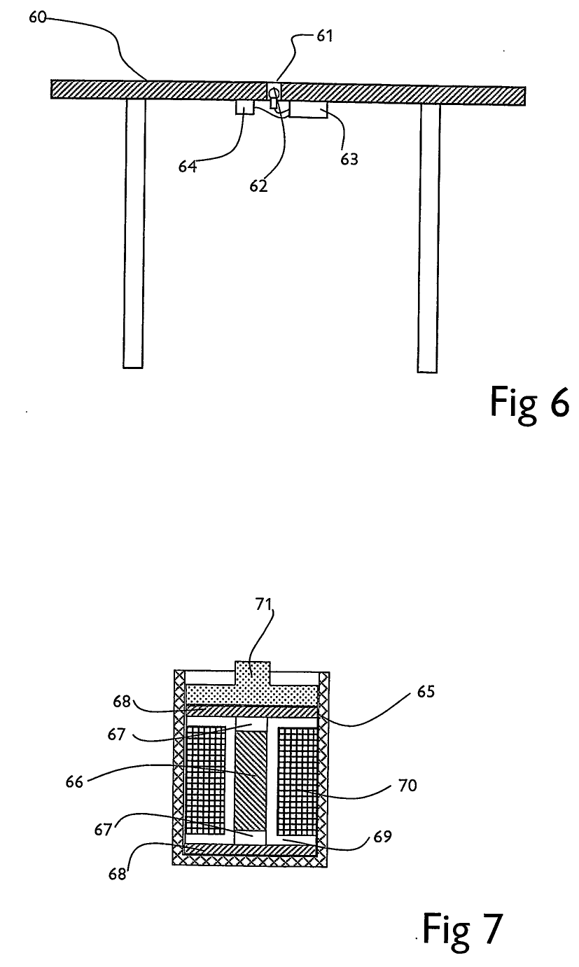

FIG. 6 illustrates a typical arrangement installed In a table top, but it will be appreciated that the system is not confined to use with tables, or indeed on horizontal surfaces. The table, for example in a bar or restaurant, has a table-top 60 provided with a central aperture 61 there through, in which is located a microphone 62 to convert the ambient noise into electrical signals which are then fed to a controller 63, mounted beneath the table-top, for example. The controller 63 suitably comprises a central processing unit programmed to perform a filtering operation which filters out speech adjacent to the microphone and then to shift the phase of the remaining signal such that, when reradiated by the transducer and table-top, as hereinafter described, it closely approximates to ambient sound, but is 180 degrees out of phase therewith, thereby substantially cancelling the sound, at least In a region close to the microphone. The effect of this will be that people sitting at the table will experience a reduction in the general ambient noise when they lean inwards towards the table's centre where the microphone is located, permitting conversation to be more easily heard. The output signal from the controller 63 is fed to a GMM transducer 64, described hereinafter in more detail with reference to FIG. 7, attached to the underside of the table so as to couple the audio signal into the table top, which then acts as a loudspeaker to radiate the sound.

Another application for the system might be installed at a serving counter or bar top, so that the barman or food server can hear orders spoken near to the microphone forming part of the system, without the need for the customer to shout to overcome general background noise.

Referring now to FIG. 7, the GMM transducer comprises a casing 65, for example of steel to provide a return magnetic path for the magnets, as hereinafter described, in which is located a rod 66 of giant magnetostrictive material (which for convenience may comprise two pieces of the GMM, end-to-end), with a non-magnetic spacer 67, for example of aluminium or a ceramic material capable of transmitting force, disposed at each end thereof to space the GMM rod 66 from a pair of disc magnets 68 of substantially greater diameter than the width of the rod. A plastics core 69 serves to locate and hold the GMM rod 66 and spacers, and carries an electromagnetic winding 70 which is connected via wires (not shown) to a separate energising signal source. A foot 71 transmits the force produced by the actuator to the desired location.

It will be understood that the invention is not limited to the use of a particular configuration of transducer, that illustrated in FIG. 7 being shown merely as an example of a usable device.

While the system is illustrated with reference to horizontal surfaces, It will be appreciated that the sound radiating surface could be vertical, for example a wall panel. It is not essential for the surface to be planar.

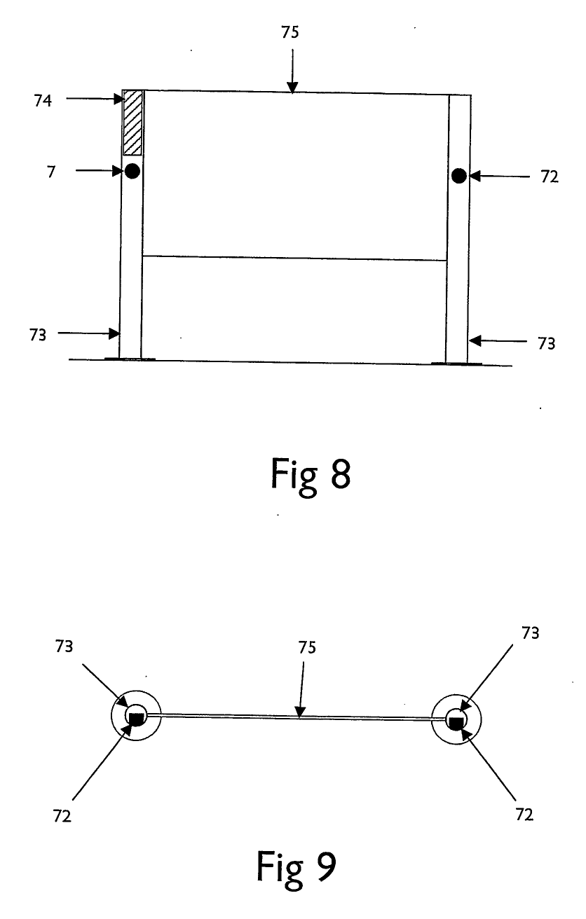

FIG. 8 shows a typical audio sign construction featuring the magnetostrictive actuator 72 mounted inside a hollow structural post 73. The audio frequency generator 74 is also mounted within the hollow post 73 along with the power supply and AGC control equipment where required. The hollow post 73 is excited by the actuator and a frequency transmitted to the rigid visual panel 75 which is attached to the post.

FIG. 9 shows the magnetostrictive actuator 72 and the method of attachment to the inside of the hollow post 73 by means of a contoured mounting bracket ensuring all generated signals are transmitted through the signs structure.

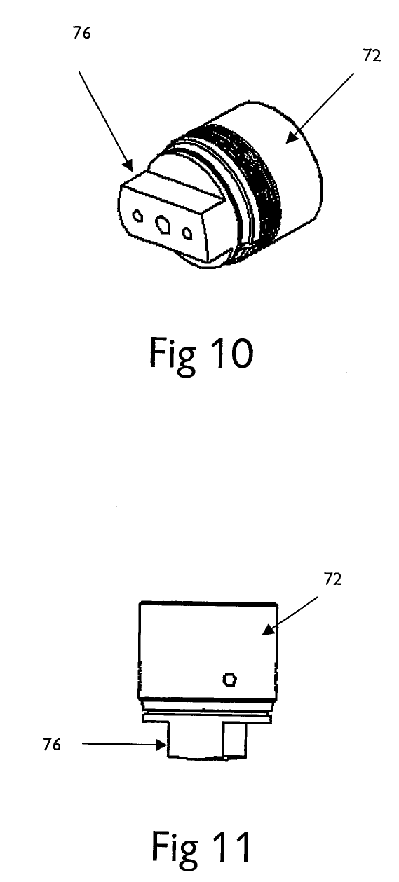

FIG. 10 and FIG. 11 show a detail illustration of the magnetostrictive actuator 72 and contoured bracket 76.

1. An audio system, comprising an audio frequency actuator coupled to a body so as to cause the body to radiate sound when an audio signal is supplied to the actuator by a controller, and an acoustic sensor coupled to said body or to an adjacent body so as to sense acoustic vibrations in said body, the sensor being connected to the controller, whereby the controller is arranged to change the audio signal supplied to the actuator according to the vibrations sensed by the sensor.

2. An audio system, comprising an audio frequency actuator coupled to a body so as to cause the body to radiate sound when an audio signal is supplied to the actuator by a controller, and an acoustic sensor coupled to said body or to an adjacent body so as to sense acoustic vibrations in said body, the sensor being connected to the controller, whereby the controller is arranged to detect predetermined characteristics of the sensed acoustic vibrations and to output an alerting signal in response thereto.

3. An audio system according to claim 1, wherein the actuator and the sensor are coupled to the same body.

4. An audio system according to claim 3, wherein the sensor is incorporated into the actuator.

5. An audio system according to claim 1, wherein the actuator is a magnetostrictive actuator.

6. An audio system according to claim 1, wherein the sensor is a piezoelectric device.

7. An audio system according to claim 1, wherein the body is rigid or flexible panel.

8. An audio system according to claim 7, wherein the panel is a ceiling panel, a wall panel, a floor panel or a window pane of a building or vehicle.

9. An audio system according to claim 1, wherein the controller is arranged to detect acoustic signals with predetermined characteristics in the output from the sensor and to supply to the actuator an audio output signal selected according to said characteristics.

10. An audio system according to claim 9, wherein the detected characteristics represent ambient noise and the output signal is a random sound signal such as white noise or pink noise adapted to provide a degree of masking of the ambient noise.

11. An audio system according to claim 9, wherein the detected characteristics represent the quality of the audio signal supplied to the actuator and the controller is adapted to correct the supplied audio signal in accordance with the detected characteristics to improve the sound radiated in the space.

12. An audio system according to claim 2, wherein the predetermined characteristics represent a damaging attack on said body.

13. An audio system according to claim 2, wherein the predetermined characteristics represent the sounds generated by a person within the space.

14. An audio system according to claim 2, wherein the alerting signal causes output of an audible and/or visible alarm.

15. An audio system according to claim 13, wherein the alerting signal is used to control the operation of lighting and/or heating within the space.

16. An audio system according to claim 13, wherein the alerting signal is used to control the supply of audio signals to the actuator.

17. A noise control system comprising a microphone associated with a rigid surface, control means connected to the microphone to detect ambient sound and arranged to generate a control signal in anti-phase to the detected sound, and a transducer supplied with the control signal, the transducer being attached to the surface so as to cause the surface to radiate sound waves which reduce the amplitude of the ambient sound in a region adjacent to the microphone.

18. A noise control system according to claim 17, wherein the transducer comprises a giant magnetostrictive material (GMM) element whose change in strain under the influence of an audio frequency magnetic field induces sound waves into the surface.

19. A noise control system according to claim 17, comprising means for filtering from the control signal speech adjacent to the microphone.

20. The noise control system according to claim 17, installed in or on a bar or counter.

21. The noise control system according to claim 17, installed in or on a bar or counter.

22. An audio system, comprising an audio frequency actuator coupled to a body which is not a flat panel so as to cause the body to radiate sound when an audio signal is supplied to the actuator by a controller.

23. An audio system according to claim 22, wherein the body is a tubular body.

24. An audio system according to claim 23, wherein the tubular body is an item of street furniture or a support therefor.

25. An audio system according to claim 24, wherein the tubular body is a supporting post for a sign.

26. An audio system according to claim 23, wherein the tubular body is non-circular in section.

27. An audio system according to claim 23, wherein the audio frequency actuator is mounted within the tubular body.

28. An audio system according to claim 23, wherein the audio frequency actuator is a transducer comprising a giant magnetostrictive material (GMM) element whose change in strain under the influence of an audio frequency magnetic field induces sound waves into the surface.

29. An audio system according to claim 2, wherein the actuator and the sensor are coupled to the same body.

30. An audio system according to claim 29, wherein the sensor is incorporated into the actuator.

31. An audio system according to claim 2, wherein the actuator is a magnetostrictive actuator.

32. An audio system according to claim 2, wherein the sensor is a piezoelectric device.

33. An audio system according to claim 2, wherein the body is rigid or flexible panel.

34. An audio system according to claim 33, wherein the panel is a ceiling panel, a wall panel, a floor panel or a window pane of a building or vehicle.
